(19) United States
(12) Patent Application Publication
(10) Pub. No.: US 2024/0094177 A1
(43) Pub. Date: Mar. 21, 2024

(54) GAS MONITORING DEVICE

(71) Applicant: Degesch America, Inc., Richmond, VA (US)

(72) Inventor: Bartek P. DREWNOWSKI, Richmond, VA (US)

(73) Assignee: Degesch America, Inc., Richmond, VA (US)

(21) Appl. No.: 18/370,584

(22) Filed: Sep. 20, 2023

Related U.S. Application Data
(60) Provisional application No. 63/408,599, filed on Sep. 21, 2022.

(51) Int. Cl. G01N 33/00

(52) U.S. Cl. CPC G01N 33/0027; G08C 17/02

(57) ABSTRACT

The present invention relates to a gas monitoring device having an enhanced gas tight base unit containing a sensor. The base unit is detachably connected to an elongated hollow rod having a perforated tip end and a base unit connection end. The base unit comprises a sensor, a sealed gap, and a parts unit having a communication module (140) containing a telemetry unit with an antenna, a processor (145) and a circuit board (150). The invention also relates to a method of monitoring the gas concentration within a fumigated commodity sample comprising inserting the tapered perforated end of a gas monitoring device into a commodity sample.

### BACKGROUND

Typically, upon harvest, an agricultural commodity is placed in jute bags, boxes and/or stored in large enclosures, such as sheds, warehouses, or silos. Agricultural commodities after harvest are often infested with insects that can consume or damage substantial amounts of the commodity.

One approach to prevent these losses is to fumigate the commodity during storage and/or immediately prior to or after shipping.

Gas fumigants have been used for decades for disinfesting closed environments infested with or suspected to be infested with insect pests such as weevils, bugs, moths and cockroaches, either mature or in various larval stages or in the form of eggs. Such fumigation is particularly used for the disinfestation of agricultural bulk commodities such as, for example, non-food commodities, processed foods, raw commodities and fresh commodities.

Phosphine (PH₃) has been a preferred gaseous fumigant for stored grain and similar particulate commodities because any residue of the fumigant will be lost or oxidized to a harmless phosphate when the grain or other commodity is processed to produce a food. Examples of the fumigation of grain with phosphine are found in the specifications of, for example, WO91/00017; U.S. Pat. Nos. 4,059,048; 4,200,657; 4,756,117; 4,812,291; 5,411,704 and 10,296,863. The entire teachings and disclosures of which are incorporated by reference herein.

The phosphine concentration pattern with the fumigation enclosure area can be influenced by, for example, temperature, air pressure and humidity. Phosphine gas concentration initially rises more or less steeply up to a maximum and from there drops asymptotically to zero at a rate which depends on phosphine losses due to leakage, decomposition or other causes. In extreme cases this may result in the phosphine concentration dropping so rapidly that complete killing of the pests, in particular their pre-adult stages, cannot be ensured. As a general rule it is preferred in phosphine fumigation to maintain lethal pesticidal gas concentrations as constantly as possible over a prolonged fumigation period. A skilled worker can refer to the teachings of U.S. Pat. No. 10,296,863 which discloses conventional calculations of air properties, boundary conditions, mass convective boundary conditions, optimization of fumigant dosage and treatment duration, how to account for gas flow within porous media, insect mortality in relation to gas levels, and various other models for estimating effective gas concentrations. The entire teachings and disclosures of U.S. Pat. No. 10,296,863 are incorporated by reference herein.

Accordingly, it would be desirable to be able to regulate the phosphine gas concentration pattern during fumigation and to maintain the desired lethal concentrations or pattern of concentrations over a prolonged period of fumigation by the controlled addition of fumigant gas.

Phosphine gas sensors serve to monitor the phosphine concentration in the enclosed fumigation area to ensure exposure sufficient to eradicate unwanted pests. Typically, phosphine gas is circulated through the stored commodity either by the natural convection currents that are present in the storage area or by active recirculation of air through the commodity using, for example, recirculation ducts. Examples can be found in, for example, U.S. Pat. Nos. 4,200,657 and 4,756,117. However, some commodities are very densely packed which creates an insulation effect whereby air/gas currents do not equally penetrate the interior of the community container. The air/gas circulation within the inner most areas of a densely packed commodity container can be significantly lower than the air/gas flow in the outer areas of a densely packed commodity container. Thus, the concentration of phosphine gas reaching the densely packed inner storage areas is often insufficient for the complete killing of pests. The industry has attempted to monitor the interior regions of a commodity container by means of gas sampling. However, this is problematic as negative pressures are created by the sampling pumps within the commodity container.

Some commercial sensors are designed to be inserted into the stream during commodity loading resulting in random placement of the sensor within the bin/truck/container. This is problematic as there is no way to retrieve the sensor to service or charge. It is also very problematic if the sensor is lost in the grain or commodity mass and is inadvertently introduced into the supply chain. A loose chip from a damaged or fragmented sensor could contaminate an entire batch of commodity resulting in substantial loss.

Thus, the need exists to monitor the supply of phosphine to all regions of the bulk-stored commodity in levels sufficient to eradicate unwanted pests. Moreover, phosphine gas is very corrosive and tends to quickly degrade or corrode electronics. Thus, the need exists for a phosphine sensor in which the electronic components are housed within an airtight sealed environment. A goal of the present invention is to provide a sensor that is able to accurately measure the gas concentration within the interior region of a bulk-stored commodity. A further goal of the present invention is to provide a sensor that is able to accurately measure the gas concentration within an ambient environment. A further goal of the present invention is to provide a sensor that is able to accurately measure the gas concentration within an ambient environment and within the interior region of a bulk-stored commodity. A further goal of the present invention is to provide a phosphine sensor device that is protected from the corrosive effects of the phosphine gas over an extended period. A further goal of the present invention is to provide a method a gaining access/space to place a sensor within the inner areas of a sealed commodity container. Upon further study of the specification and appended claims, further goals, objects and advantages of this invention will become apparent to those skilled in the art.

### BRIEF SUMMARY OF INVENTION

One embodiment of the present invention relates to a gas monitoring device comprising a perforated tip detachably connected to an enhanced gas tight housing unit having a rod end and a base end. The base end comprises a sensor, a sealed gap (155) between the bottom (111) of the sensor and a parts unit (139) containing a communication module (140) having a telemetry unit with an antenna, a processor (145), a circuit board (150) and an internal on/off switch. The rod end is narrower than the base end. Preferably, the elongated rod end of the housing is between 12 to 36 inches long. Preferably, the rod end is between 0.5 to 1 inch in diameter. Preferably, the perforated tip is between 0.5 and 2 inches long. Preferably, the base end is 4 to 10 inches wide and 2 to 6 inches tall. Preferably, the housing unit is made of a shock absorbing plastic.

A further embodiment of the present invention relates to a two piece commodity probe comprising a hollow outer sleeve with a tapered wall at a first end and a collar on a second end and a removable inner core with a spike on a first end and a collar on a second end. The inner core fits within the hollow outer sleeve and the spike extends beyond the tapered wall at the first end of said outer sleeve.

A further embodiment of the present invention relates to a method of monitoring the gas concentration within a fumigated commodity sample comprising inserting a two piece commodity probe into a commodity sample. The method advantageously does not require external gas sampling pumps. External gas sampling is often problematic and results in unreliable concentration data. The present method uses a two piece commodity probe comprising a hollow outer sleeve with a tapered wall at a first end and a collar on a second end and a removable inner core with a spike on a first end and a collar on a second end. The inner core fits within the hollow outer sleeve and the spike extends beyond the tapered wall at the first end of said outer sleeve. After placement in the commodity, the inner core is removed and a gas monitoring device is inserted into the hollow outer sleeve. The preferred gas monitoring device comprises a perforated tip connected to a gas tight housing unit having a rod end and a base end. The base end comprises a sensor and a sealed gap between the bottom of the sensor and the parts unit, a battery capable of wireless charging and an internal on/off switch. Connecting wire connects the sensor to the circuit board. The rod end is narrower than the base end.

Preferably, the gas monitoring method of the present invention measures phosphine gas concentration in parts per million. The data is collected and transmitted via a telemetry based communication unit at preset intervals and sent to cloud storage and/or cell phone.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 depicts an embodiment of a gas monitoring device according to the present invention (not to scale).

FIG. 2 depicts an embodiment of a gas monitoring device according to the present invention inserted with an enclosed environment containing stored commodities (not to scale).

FIG. 3 depicts an embodiment of a gas monitoring device according to the present invention.

FIG. 3a depicts an embodiment of a gas monitoring device according to the present invention which is free-standing when the bottom (136) of the base end (130) is placed on a solid support.

FIG. 3b depicts an embodiment of a gas monitoring device according to the present invention where the elongated hollow rod is detached from the base end (130).

FIG. 3c depicts a detail close up view of the tip end of an embodiment of an elongated hollow rod according to the present invention. Perforations are spaced along the top portion of the tip end of the elongated hollow rod.

FIG. 3d depicts a detail close up view of o-ring seals within the connection between the base unit and the elongated hollow rod.

FIG. 4 is cross section view of an embodiment of the present invention showing the attachment of the base unit connection end (124) of the hollow rod to the base unit. The external on/off activation switch (135) can be seen.

FIG. 5 is a top side view of an embodiment of the present invention showing the attachment of the base unit connection end (124) of the hollow rod to the base unit. The external on/off activation switch (135) activates the internal magnetic on/off switch.

FIG. 6 depicts an embodiment of a gas monitoring device according to the present invention in which the sensor is in the tip end.

FIG. 7 depicts an embodiment of a two piece probe device according to the present invention.

FIG. 8 depicts an embodiment in which a two piece probe device according to the present invention inserted into a commodity container.

FIG. 9 depicts an embodiment in which the outer sleeve of the two piece probe device according to the present invention remains in the commodity container and an embodiment of a gas monitoring device according to the present invention is inserted into the outer sleeve.

FIG. 10 depicts an embodiment of a gas monitoring device according to the present invention placed with an enclosed environment containing stored commodities. The unit is free-standing when the bottom (36) of the base end (30) is placed on a solid support.

FIG. 11A depicts a sensor tip embodiment of the gas monitoring device according to the present invention.

FIG. 11B depicts a sensor tip embodiment of the gas monitoring device according to the present invention.

FIG. 11C depicts a sensor tip embodiment of the gas monitoring device according to the present invention.

FIG. 11D depicts a sensor tip embodiment of the gas monitoring device according to the present invention.

FIG. 11E depicts a sensor tip embodiment of the gas monitoring device according to the present invention.

FIG. 12 is a cut away view of an embodiment of the present invention showing a two part base unit on top of a charging docking base (153). The base unit has a top half (151) and a bottom half (152). A sensor (110) is located within in the uppermost potion of the base unit, a sealant filled gap (155) is located between the bottom of the sensor (110) and the top of the parts unit (139). A rechargeable battery (33) and an induction coil (158) are located below the parts unit. A docking base (153) contains a wireless charger (34) and a second induction coils. Together, the two coils make up an electrical transformer. The external on/off activation switch (135) activates the internal magnetic on/off switch.

FIG. 13 is a cut away view of an alternative embodiment of the present invention showing a two part base unit. The base unit top half (151) and bottom half (152) contain silicone seals (157) around the perimeter and Viton seals (161) on the top half.

FIG. 14 depicts an alternative embodiment of the present invention showing two elongated hollow rods attached to the base end (120, 162).

### DETAILED DESCRIPTION

As used herein the term “perforations” or “perforated” refers to openings which allows airflow to reach the interior of the elongated hollow rod. They can be any size and shape (e.g., slots, holes, ovals, squares) which allow air into the interior of the elongated hollow rod to reach the sensor in the base unit. Preferably, the perforations are within the tip end of the elongated hollow rod which is about the top 50% of the elongated hollow rod.

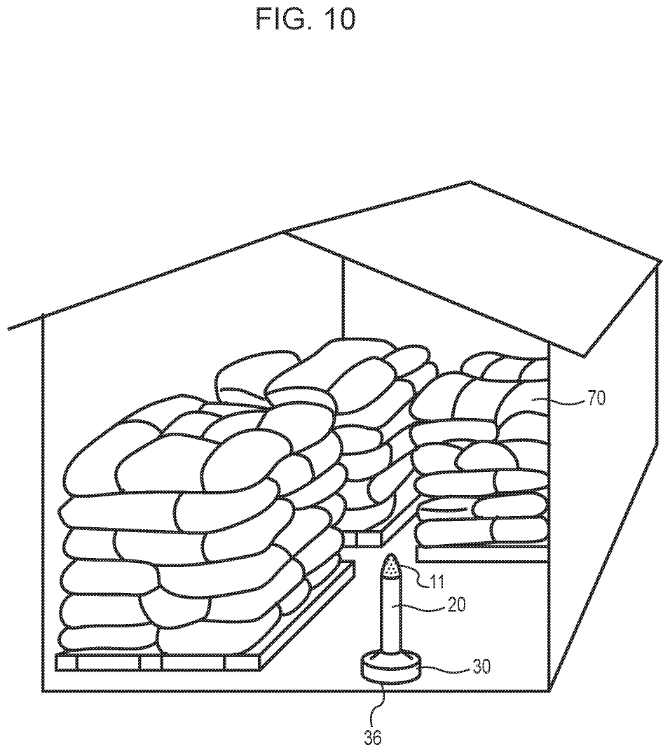

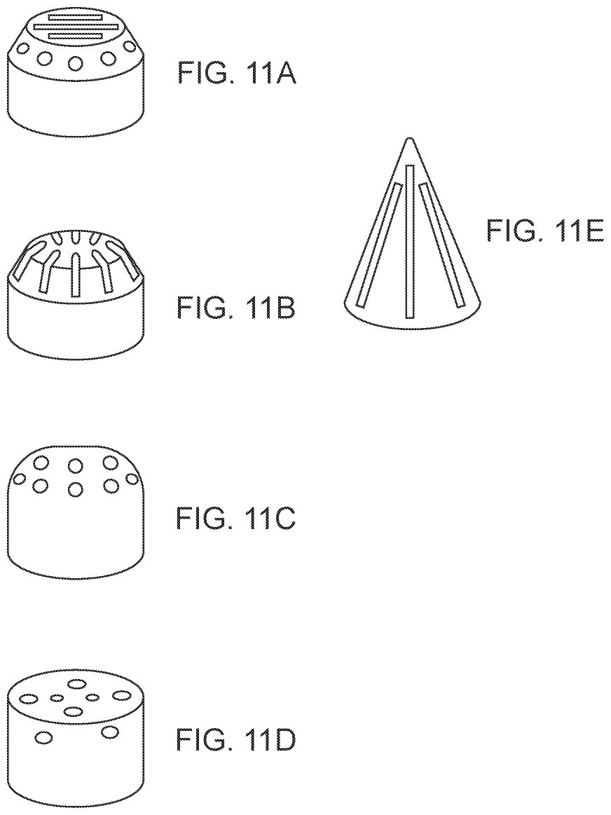

The tip perforations can be any size and shape (e.g., slots, holes, ovals, squares) which allow air to reach the sensor. Several contemplated variations are depicted in FIGS. 11A, 11B, 11C, 11D and 11E.

As used herein the term “Commodity container” refers to an enclosed environment containing stored commodities (e.g., vertical storage, tanks, flat storage (loose construction), farm bins, bunkers, tarped ground storages, railcars, barges, ship-holds, mills, warehouses, chambers, or silos). Commodity container can also include sub-containers stored with a larger enclosed environment such as, for example, cartons, wooden barrels, jute bags, woven bags, woven poly, supersack, bales, mesh bags, paper bags and/or plastic/poly bags.

Typical commodities requiring fumigation include, for example, non-food commodities, processed foods, raw commodities and fresh commodities.

Non-food Commodities include, for example, processed or unprocessed cotton, wool and other natural fibers or cloth, clothing; straw and hay; feathers, human hair, rubberized hair, vulcanized hair, mohair, leather products, animal hides and furs, tobacco, tires (for mosquito control), wood, cut trees, wood chips, wood products, bamboo products, paper, paper products, psyllium seed, psyllium seed husks, dried plants, flowers, seeds (such as grass seed, ornamental herbaceous plant seed and vegetable seed).

Processed food commodities include, for example, processed candy and sugar, cereal flours and bakery mixes, cereal foods (including cookies, crackers, macaroni, noodles, pasta, pretzels, snack foods and spaghetti), processed cereals (including milled fractions and packaged cereals), cheese and cheese byproducts, chocolate and chocolate products (such as assorted chocolate, chocolate liquor, cocoa, cocoa powder, dark chocolate coating and milk chocolate products), processed coffee, corn grits cured meat products, dried fish, dates, figs, dried eggs, egg yolk solids, dried milk, dried powdered milk, non-dairy creamers, non-fat dried milk, dried or dehydrated fruits (such as apples, dates, figs, peaches, pears, prunes, raisins, citrus and sultanas), processed herbs, spices, seasonings, condiments, malt, processed nuts (such as almonds, apricot kernels, brazil nuts, cashews, filberts, macadamia nuts, peanuts, pecans, pistachio nuts, walnuts and other processed nuts), processed oats (including oatmeal), rice (brewer's rice, grits, enriched and polished), soybean flour and milled fractions, processed tea, dried and dehydrated vegetables (such as beans, carrots, lentils, peas, potato flour, potato products and spinach), yeast (including primary yeast) wild rice and other processed foods.

Raw Commodities include, for example, almonds, animal feed & feed ingredients, barley, brazil nuts, cashews, cocoa beans, coffee beans, corn, cottonseed, dates, filberts, flower seeds, grass seeds, legume vegetables (dried), millet, oats, peanuts, pecans, pistachio nuts, popcorn, rice, rye, safflower seeds, sesame seeds, sorghum, soybeans, sunflower seeds, triticale, vegetable seeds, walnuts and/or wheat.

Fresh commodities include, for example, alfalfa, avocado, banana (including plantains), cabbage, citrus, citron, dill, eggplant, endive, grapefruit, kumquat, legume vegetables (succulent), lemon, lettuce, lime, mango, okra, orange, papaya, pepper, persimmon, pimento, salsify tops, sweet potato, tangelo, tangerine and/or tomato.

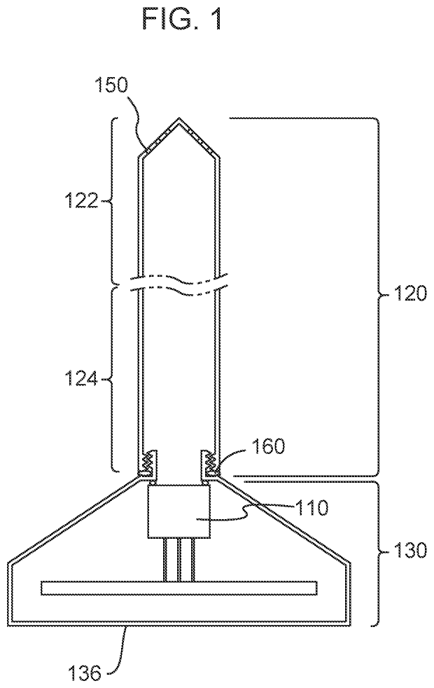

FIG. 1 depicts a preferred embodiment of the present invention (not to scale). The gas monitoring device comprises a gas tight base unit (130) containing a sensor (110). The base unit is detachably connected to an elongated hollow rod (120) having a tapered tip end (122) and a base connection end (124). The base unit comprise a sensor (110), data processor, a telemetry unit, a battery with wireless charging and an internal on/off switch. Preferably, the elongated hollow rod end is between 12 to 36 inches long. Preferably, the perforated tip end is in the shape of a spike. Preferably, the base end is 4 to 10 inches wide and 2 to 6 inches tall. Preferably, the housing unit is made of a shock absorbing plastic. In certain embodiments the base unit may contain one or more additional sensors to detect various other environmental conditions.

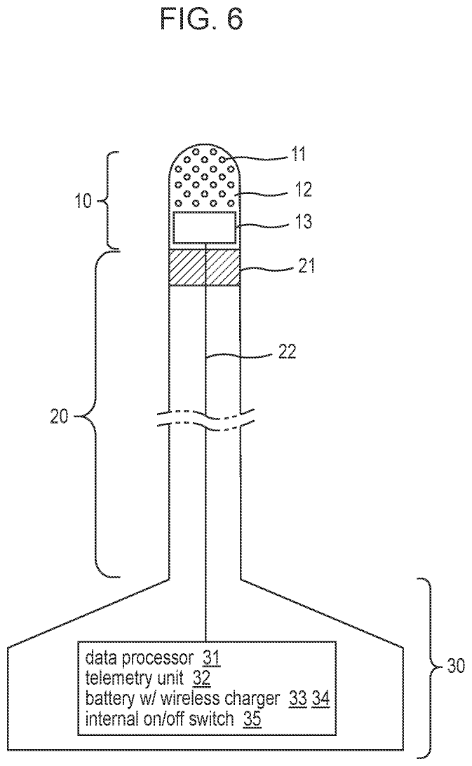

FIG. 6 depicts another preferred embodiment of the present invention, a gas monitoring device comprising a perforated sensor tip (10) detachably connected to a gas tight housing unit having a rod end (20) and a base end (30). The base end houses a data processor (31), a telemetry unit (32), a power supply (e.g., battery (33) with wireless charging (34)) and an internal on/off switch (35). The sensor tip (10) contains a gas sensor (13) and optional filter media (12). In certain embodiments the sensor tip may contain the one or more additional sensors to detect various other environmental conditions. Preferably, the gas sensor is a phosphine high range sensor (0-2000 ppm) located within a rounded or domed perforated tip. However, the top of the tip may be flat, domed, pointed etc. The perforations may be any shape which allow air/gas to circulate and reach the sensor. FIGS. 11A-11E depicts several contemplated variations for the shape of the sensor tip and the shape of the openings within the sensor tip. For example, the perforations may be slots, holes, ovals, squares etc. The perforations may also be a combination of shapes. In a preferred embodiment filter media covers the sensor to protect the sensor from dust or particulate matter. The rod end (20) contains a gas tight plug (21) through which a connecting wire (22) operably connects the sensor (13) to the data processor (31) in the base end (30) of the housing unit. The rod end (20) is narrower than the base end (30). Preferably, the elongated rod end of the housing unit is between 18 to 32 inches long. Most preferably, the elongated rod end of the housing unit is between 22 to 25 inches long. The rod end (20) is generally between 0.5 to 1.5 inches in diameter. Preferably, the rod end is between 0.5 to 1 inches in diameter. Most preferably, the rod end is between 0.5 to 0.8 inches in diameter. Generally, the perforated sensor tip (10) is between 0.3 and 2 inches long. Preferably, the perforated sensor tip is between 0.4 and 1.25 inches long. Most preferably, the perforated sensor tip is between 0.5 and 1 inches long. The device can advantageously measure phosphine concentrations from ambient air or commodity phosphine concentrations from within the inner areas of a fumigated commodity container without the use of external mechanical sampling pumps.

The parts unit contains a telemetry unit (32) which transmits collected data via wireless data transfer mechanisms (e.g., using radio RFID, ultrasonic, infrared systems, cellular telephone networks (e.g., GSM networks using SMS)). Preferably, the antenna is located toward the outer periphery of the parts unit in the region closest to the base unit housing.

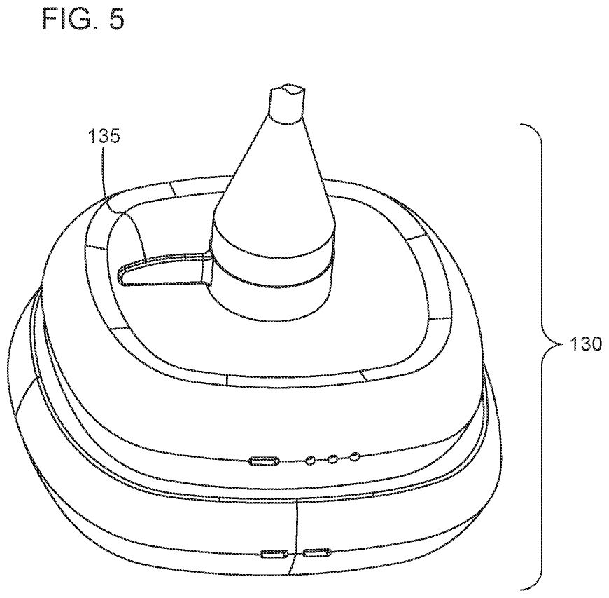

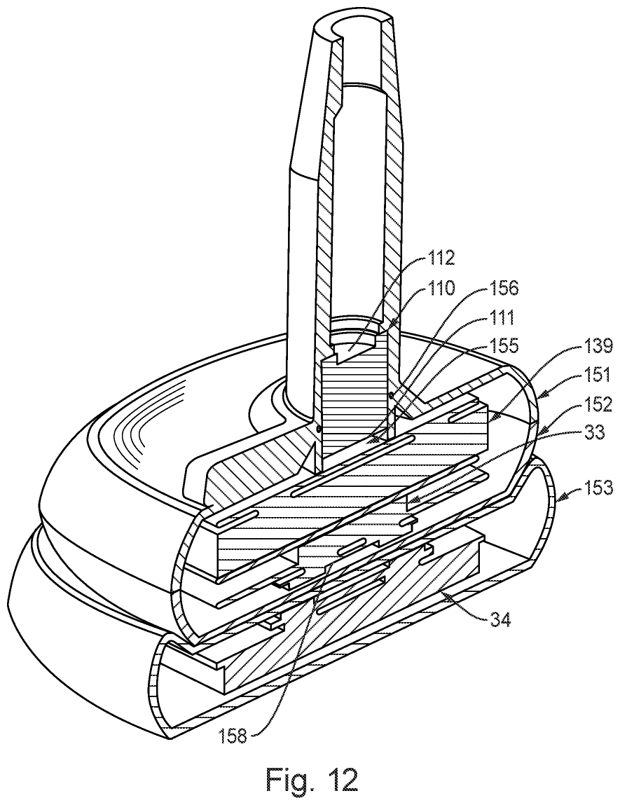

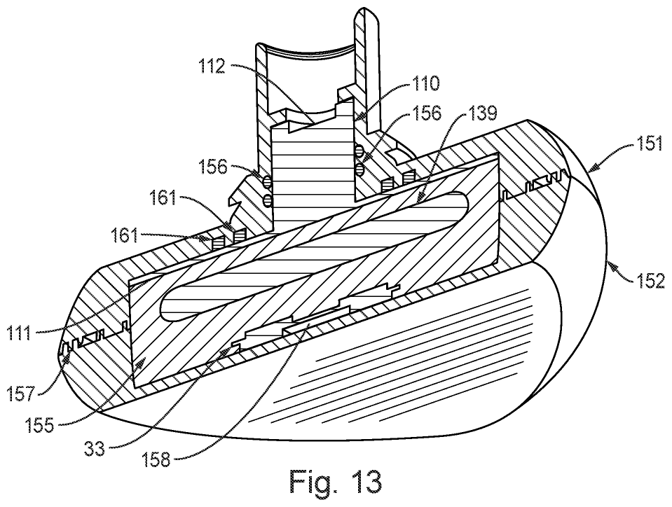

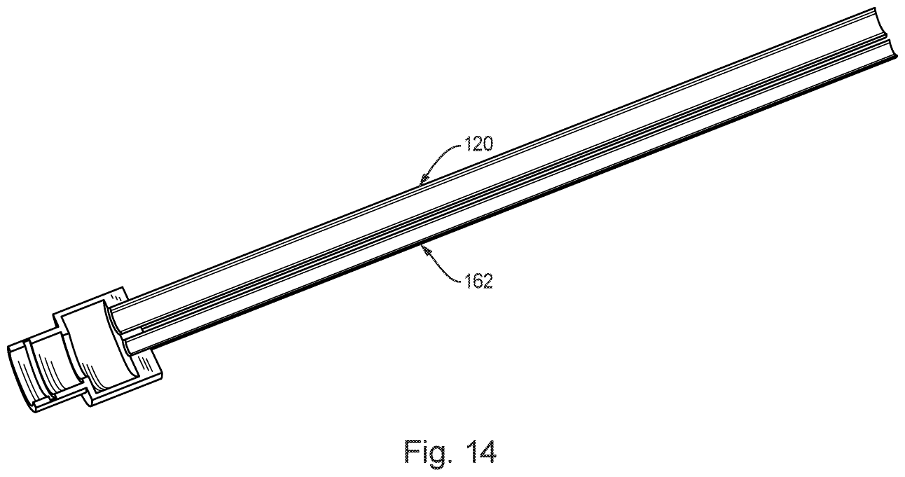

In preferred embodiments the gas monitoring device stands between about 22 to 38″ tall. The tapered end of the elongated rod is perforated which allows for air/gas movement within the space. The elongated rod is detachably removable from the base. Thus, in certain environments it is desirable to place a removable filter medium within the rod so that it sits above, or adjacent the sensor contained within the base. The filter will protect the sensor from particulate matter and solid dust particles. The sensor operably connects to the circuit board located in parts unit of the base end (130). The base end is preferably 4″ to 10″ wide and 2″ to 6″ tall with a wall sloping inward towards the juncture with the rod end forming a cone shaped round base. In a preferred embodiment the base is adapted to support the device in a free-standing upright position such as depicted in FIG. 10. In another preferred embodiment, the base is adapted to rest upon a charging docking base (153) which is capable of charging the wireless battery in the base unit, such as depicted in FIGS. 5 and 12. The base end will house electronics (e.g., sensor, sealant gap, a battery, on/off switch, induction coil, data processor (145), a communications module with a telemetry unit and an antenna, and a power indicator). During manufacturing of the device the components are operably connected and installed within the base unit. After installation a bottom plate will be sealed into position on the bottom of the base end. In another preferred embodiment, shown in FIG. 12, the base is comprised of a top half (151) and a bottom half (152) which are sealed together after installation of the components. In preferred embodiments the housing of the device will be made of molded shock absorbing plastic. It is also preferred that the housing be of high visibility colors such as, for example, orange. The housing may also contain high visibility markings such as reflective strips or patches. Preferably, high visibility markings are placed on the base unit.

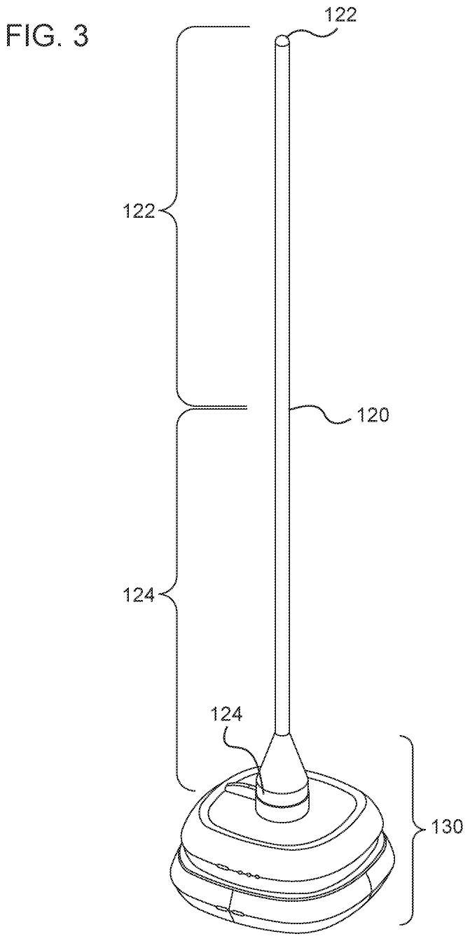

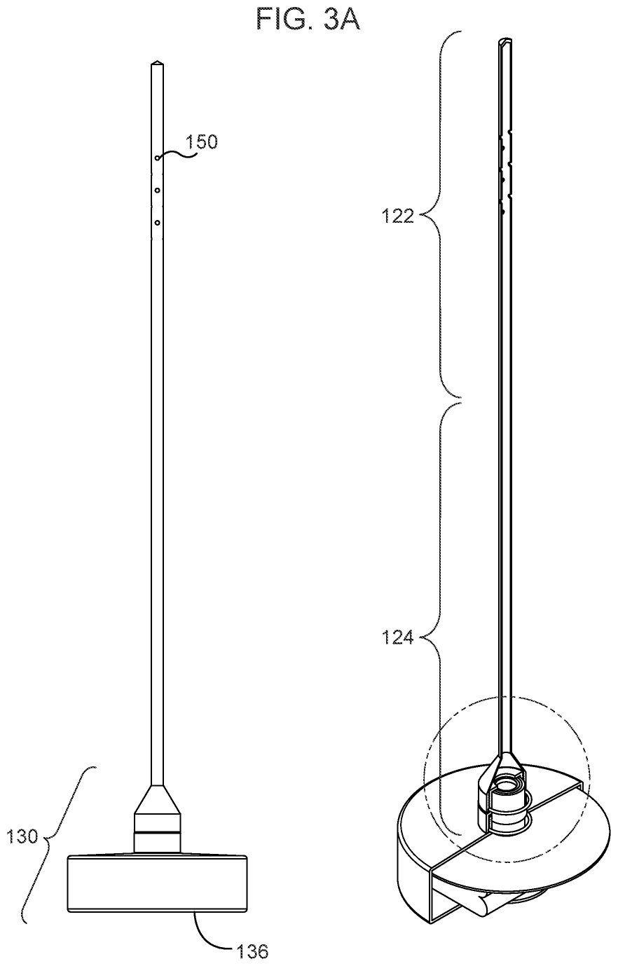

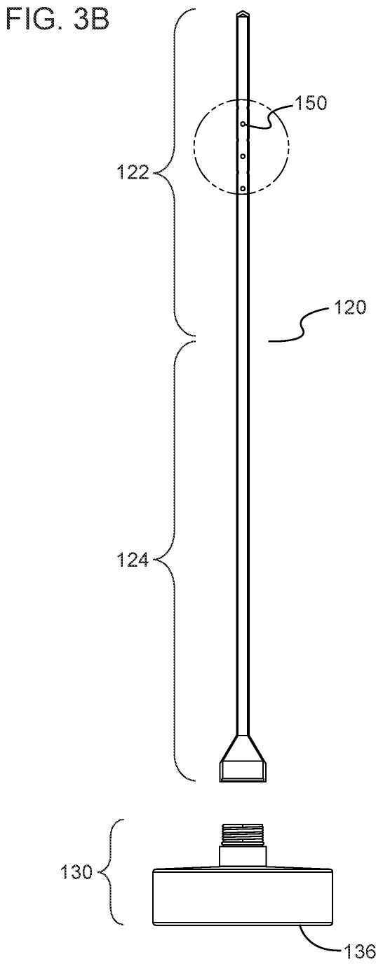

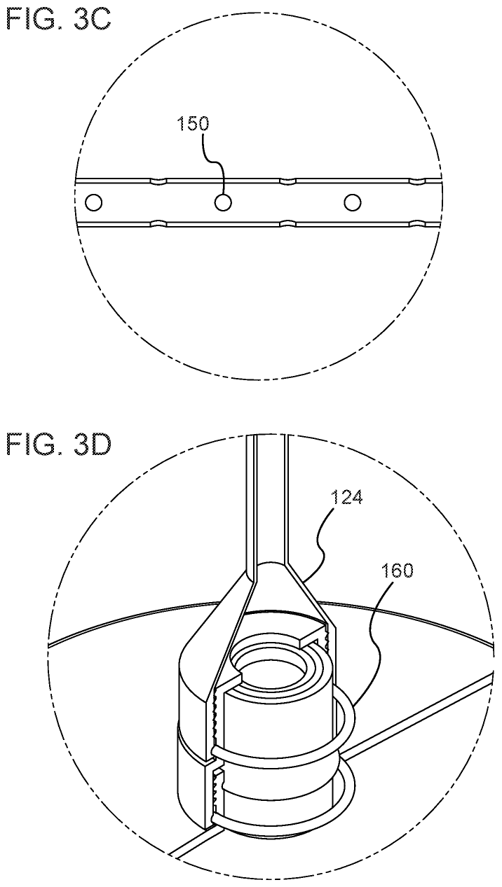

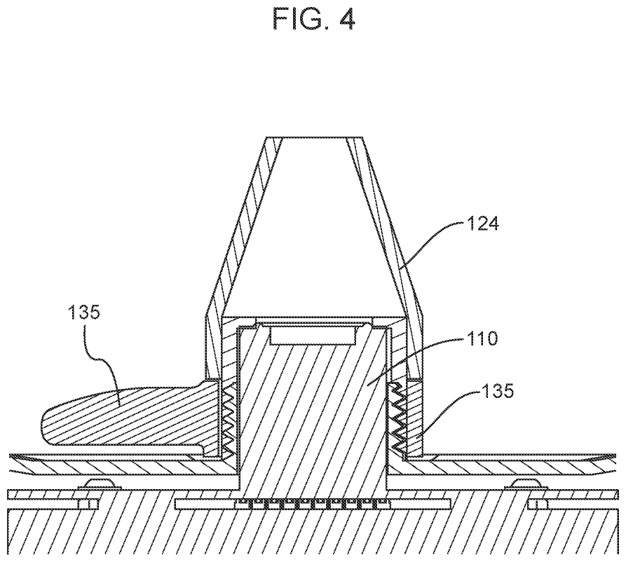

Generally, the base end is 4 to 10 inches wide and 2 to 6 inches tall. Preferably, the base end is 6 to 9 inches wide and 3 to 5 inches tall. Most preferably, the base end is 7 to 8.5 inches wide and 3.5 to 5 inches tall. It should be recognized that the base unit width is sufficient to support the housing unit in an upright position when placed on a support such as a floor. See, for example, FIGS. 3 and 10. Preferably, the housing unit is made of a shock absorbing plastic. In certain embodiments the base unit is encased in a shock absorbing outer protective wrap/shell such as, for example, a silicone or rubber skin. Preferably, the protective wrap/shell is high visibility. Preferably, all hardware contained within the device is intrinsically safe (i.e., UL/IP67/CE/ATEX approved).

The base end of the device houses a data processer which may also contain internal memory. The data processor is configured to execute instructions in the memory and to read and write data to and from the memory. Preferably, the base unit contains enough memory to capture, for example, at least 30 days of data.

The base end also contains a parts unit (139) containing a communication module (140) having a telemetry unit with an antenna. The telemetry unit sends data preferably via non-Wi-Fi or Bluetooth means. Telemetry based communications such as SMS/GMS can work without the internet on mobile devices allowing push notifications in cases where the gas concentration thresholds dip above and/or below a preset level. The device is capable of worldwide communication. The telemetry unit includes an antenna operably connected to a radio transmitter configured to wirelessly transmit data. Preferably, the antenna is located in the outer periphery of the parts unit (the edge of the parts unit closest to the base unit housing). The telemetry device can send data to cloud storage where it can be processed into visually presentable data and graphic depictions of the fumigation process. The telemetry unit can be adapted to transmit gas (e.g., Phosphine) ppm data at selected preset intervals e.g., every 2, 4, 6, 8, 12, 24 hours. To save on battery life, the device may operate in a sleep mode in between selected intervals. The device may be a configured for a higher power usage awake mode and a lower power usage sleep mode thus extending battery life.

The base unit also houses a power supply e.g., battery (133) which may be operably coupled to the memory chip, the data processor, the radio transmitter, on-off switch and/or the sensor. Preferably, as the base unit (130) is sealed the power supply is a battery capable of wireless charging. Wireless charging, is also referred to as induction charging, and consist of two primary induction coils. One is housed in an external ‘charging base’, which is also commonly referred to as the ‘mat’. The external charging mat is responsible for generating an alternating current (AC) from within the mat. The other induction coil is located in the bottom half of the base device. Together, these two coils make up an electrical transformer.

When the base device is placed on a charging mat, it receives and harvests energy from a magnetic field, and uses it to power or recharge the battery.

The base unit may also optionally contain an LED power indicator, an ON/OFF indicator and a charge indicator. Moreover, as the unit is sealed the base unit also contains a magnetic on/off switch operably coupled to the power supply. The magnetic switch allows the sealed device to be turned on/off without an external connection into the sealed housing. A FOB associated with the unit may be used to activate the magnetic on/off switch. A cleaning tool may also be included with the FOB to allow cleaning of the perforations in the sensor tip.

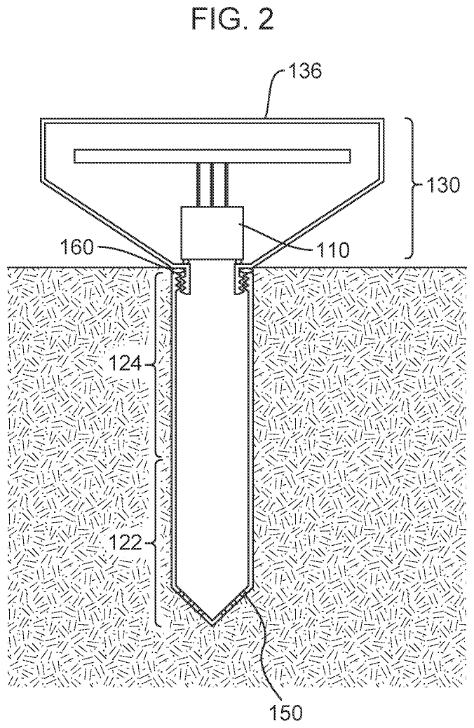

FIG. 2 depict a method of monitoring the gas concentration within a fumigated commodity sample without the use of external mechanical sampling pumps. FIG. 2 depicts a device according to the present invention inserted into a commodity container. The force required to insert the device will depend on the commodity and how densely the commodity is packed within the container. Some commodities such as, for example, a densely packed tobacco may require a hammer to insert the device into the commodity. In some commodities such as, for example, corn kernels, the device can be placed with hand pressure alone. The outer surface of the device will be in direct contact with the commodity and therefore should be made of a recognized food contact material (FCM). FCMs must be sufficiently inert so that they do adversely affect consumer health nor influence the quality of the food. Preferably, the outer sleeve is stainless steel.

FIG. 12 depicts a preferred embodiment of the present invention in which the sensor (110) is located in the top of the body of the base unit with the sensor top (112) facing upwards so as to be in contact with the ambient air and the sensor bottom (111) facing a sealed gap (155). The sealant filled gap (155) is located between the bottom of the sensor and the parts unit which contains the circuit board, data processor and telemetry unit. Preferably, the antenna of the telemetry unit is located in the outer periphery of the parts unit (the portion of the parts unit closest to the housing of the base unit).

Due to the corrosive nature of phosphine gas the electronic components housed within the base (in particular the antenna) must be protected in an airtight sealed environment. The sensor is sealed to the housing with rubber O-rings (156) and the base unit openings are sealed. Sealing of sensor with o-rings and providing an airtight seal to the base unit often does not provide sufficient long term protection from corrosion as it was discovered that the sensor itself is slightly permeable to the ambient air. The permeability of the sensor allows corrosive phosphine gas to reach the interior of the base unit resulting in early degradation of the of the base unit components with long term use. However, placement of additional sealant within the base unit itself needs careful consideration to protect against device failure. Sealant could transfer unexpected force to the data processor and telemetry unit, which could result in mechanical failure or be too insulating and cause thermal failure. Moreover, a suitable sealant must itself be non-corrosive to the interior components of the base unit. The above mentioned failure concerns were addressed, in part, by placing a gap filled with a suitable sealant between the bottom of the sensor and the parts unit (139) containing a communication module (140), a processor (145) and a circuit board (150). Most preferably, the gap sealant is a RTV silicone. The antenna is located on the periphery of the parts unit. The leads from the sensor to the circuit board run through the gap.

Preferably, the base unit is composed of two housings halves. The top half (151) and bottom half (152) are joined together with a chemical resistant adhesive/sealant. The adhesive/sealant used to join the top and bottom of the housing may be the same as or different from the adhesive/sealant used in the sealant gap between the bottom of the sensor and the circuit board. Preferably, the adhesive/sealant is a urethane, polysulfide, latex or silicone based sealant such as, for example, a silicone rubber. Preferably, the silicone adhesive/sealant has good flow-ability, low shrinkage, exhibits a high-temperature resistance, is acid and alkali resistant, and exhibits ageing resistance. The sealant is selected to be non-corrosive to the interior components of the base unit and with careful consideration of thermal insulating properties and aging resistance. Most preferably, the sealant is a RTV silicone (room-temperature-vulcanizing silicone). RTV silicones typically have a quick cure time and are commercially available as one-component products or mixed from two-components (a base and curative). RTV adhesive/sealant also allows for easier separation of the two base unit housing halves if repair or adjustments are required. In a preferred embodiment, sealant is also placed in a tongue groove that runs around the joining edge of the base unit halves. In certain embodiments the sealant may also applied to screws, which join the two housings halves of the base unit (top and bottom).

Additional protection is provided by application of a conformal coating to the base unit interior components. A conformal coating is a thin polymeric film, which conforms to the contours of the parts unit to protect the components against moisture, dust, chemicals, and temperature extremes. Conformal coatings are breathable. These coatings are not sealants. Conformal coatings also prevent current bleed between closely positioned components. Typically, conformal coatings are 10-250 μm thick.

Coatings can be applied in a number of ways, including brushing, spraying, dispensing and dip coating. Furthermore, a number of materials may be used as a conformal coating, such as acrylics, silicones, urethanes and parylene. Humiseal 1A33™ is a preferred commercially available conformal coating available from CHASE Corp. in Pittsburgh PA.

Telemetry based communications such as SMS/GMS can work without the internet on mobile devices allowing push notifications in cases where the gas concentration thresholds dip above and/or below a preset level. The device is capable of worldwide communication. The telemetry unit includes an antenna operably connected to a radio transmitter configured to wirelessly transmit data. The parts unit contains an antenna and a tuner circuit. To avoid overheating and distortion the antenna is preferably located on the outer periphery of the parts unit (towards the base unit housing).

Probe

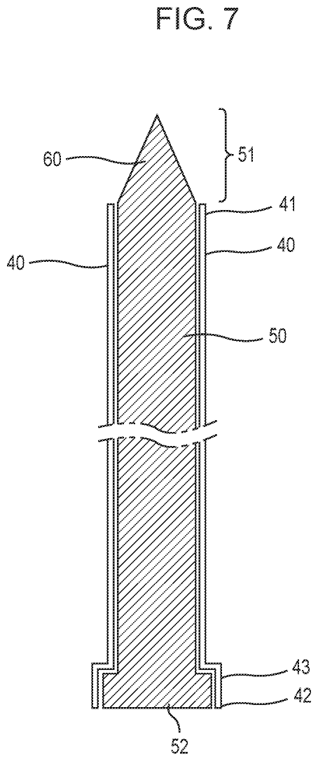

Some embodiments are device is designed to be compatible with a 2-piece stainless probe unit used for the optional mode of inserting the device into a commodity. FIG. 7 depicts an embodiment of a two piece probe device according to the present invention. The probe will be a 2-piece stainless steel device approximately 20″ to 30″ in length. The outer sleeve (40) is a hollow tube with a tapered wall on one end and optionally a stainless steel round collar (43) on the second end of the outer sleeve (42). The tapered wall of the first end of the outer sleeve (41) allows the device to be more easily inserted into a commodity. The internal removable inner core (50) includes spike component (60) on the first end (51). The spike is typically slightly longer than the outer sleeve. The sharp pointed tip allows the spike to more easily penetrate the commodity. The inner core optionally contains a round collar welded on the other end. Once both pieces are inserted into the commodity packaging, the inside core will be removed leaving behind the hollow outer sleeve. The inside diameter of the outer sleeve will be just large enough to allow the insertion of the rod end of the measuring device.

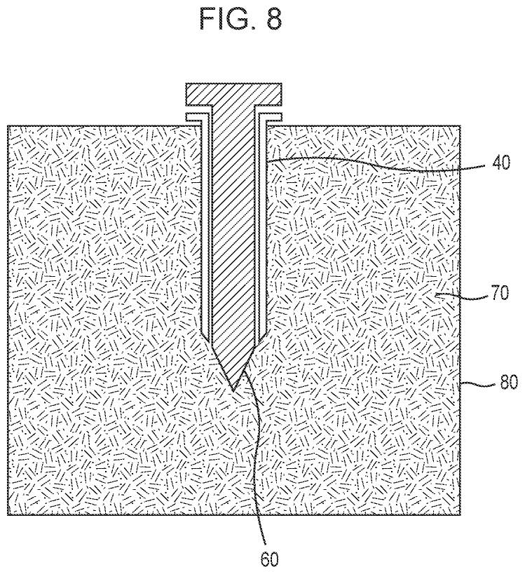

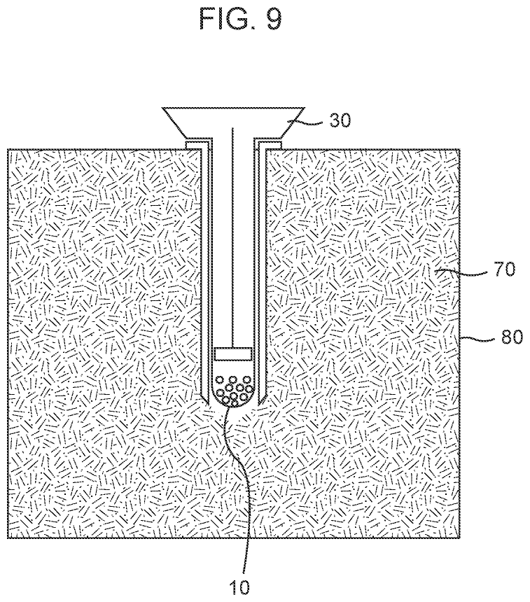

FIGS. 8 and 9 depict a method of monitoring the gas concentration within a fumigated commodity sample without the use of external mechanical sampling pumps. FIG. 8 depicts a two piece probe device according to the present invention inserted into a commodity container. The force required to insert the two piece probe will depend on the commodity and how densely the commodity is packed within the container. Some commodities such as, for example, a densely packed tobacco may require a hammer to insert the two piece probe into the commodity. In some commodities such as, for example, corn kernels, the two piece probe can be placed with hand pressure alone. The outer sleeve of the two piece probe will be in direct contact with the commodity and therefore should be made of a recognized food contact material (FCM). FCMs must be sufficiently inert so that they do adversely affect consumer health nor influence the quality of the food. Preferably, the outer sleeve is stainless steel.

FIG. 9 depicts an embodiment in which the outer sleeve of the two piece probe device according to the present invention remains in the commodity container and a gas monitoring device according to the present invention is inserted into the outer sleeve. Preferably, the sensor tip (10) does not extend beyond the first end of the outer sleeve so as not to contact the commodity. In certain embodiments where the sensor tip does extend beyond the outer sleeve the sensor tip is be made of a recognized food contact material (FCM). The sensor is now positioned to monitor the gas concentrations within the inner areas of the insulated commodity without the need for problematic and unreliable external gas sampling.

End Use Software

The device includes software for fresh air calibration. Preferably, the unit zeros itself out when turned on. The device also includes calibration software which follow industry guidelines for gas concentration.

The base unit is adapted to transmit gas (e.g., Phosphine) ppm data at selected preset intervals e.g., every 2, 4, 6, 8, 12, 24 hours. The data, along with a time stamp, can be processed and accessed through a dashboard for viewing a graphical evaluation and/or reports of the fumigation concentration over time. Prior to fumigation, each job will be assigned a unique name (location and date) and each gas monitoring device is assigned a unique code (e.g., 4-digit unit ID). The unique location and unique device code associated with that fumigation are entered via the dashboard and named with a relevant name (e.g., warehouse number, silo number, container number, tarp number, etc.). During the fumigation, if a concentration received is below a preset ppm level, the software will send out an email and/or SMS notification to alert a predetermined group. Once the fumigation is over, the software will generate a post fumigation graph of phosphine concentrations in ppm vs. time in hours. The software allows access for guests invited to view the results of only that specific fumigation and the specific base units associated with it. Once the fumigation is over, the job will be finalized, and base units “released” from assignment.

Without further elaboration, it is believed that one skilled in the art can, using the preceding description, utilize the present invention to its fullest extent. The preferred specific embodiments are, therefore, to be construed as merely illustrative, and not limitative of the disclosure in any way whatsoever.

From the foregoing description, one skilled in the art can easily ascertain the essential characteristics of this invention and, without departing from the spirit and scope thereof, can make various changes and modifications of the invention to adapt it to various usages and conditions.

We claim:

1. A gas monitoring device comprising:
a gas tight base unit containing:
a) a sensor (110) having a top (112) and a bottom (111),
b) a sealant filled gap (155),
c) a parts unit (139) containing a communication module (140) having a telemetry unit with an antenna, a data processor (145) and a circuit board (150),
d) a battery (33)
e) an internal on/off switch
and
f) an induction coil (158)
said base unit is detachably connected to an elongated hollow rod having a perforated tip end and a base unit connection end,
and
wherein the sealant filled gap is between the bottom of said sensor and said parts unit.

2. A gas monitoring device according to claim 1, wherein said elongated hollow rod is between 12 to 36 inches long.

3. A gas monitoring device according to claim 1, wherein elongated hollow is between 0.5 to 1 inch in diameter.

4. A gas monitoring device according to claim 1, wherein said base end is 4 to 10 inches wide and 2 to 6 inches tall.

5. A gas monitoring device according to claim 1, wherein said housing unit is made of a shock absorbing plastic.

6. A gas monitoring device according to claim 1, wherein the sealant in the sealant filled gap is a RTV silicone

7. A method of monitoring the gas concentration within a fumigated commodity sample comprising:
inserting a tapered perforated end of a gas monitoring device into a commodity sample,
said a gas monitoring device comprising
a gas tight base unit having a top side and a bottom side, said top end is detachably connected to a base unit connection end of an elongated hollow rod having said tapered perforated end,
wherein said base unit comprises a sensor, a sealant filled gap, a communication module (140) having a telemetry unit with an antenna, a processor (145), a circuit board (150), a battery, an induction coil and an internal on/off switch.

8. A method according to claim 7, wherein said gas is phosphine and parts per million data is collected and transmitted via telemetry based communication unit at preset intervals.

9. A method according to claim 7, wherein said telemetry unit sends data to cloud storage.

10. A method according to claim 7, wherein said elongated hollow rod further comprises a filter.

11. A method according to claim 7, wherein said elongated hollow rod is between 12 to 36 inches long.

12. A method according to claim 7, wherein said elongated hollow rod is stainless steel.

13. A method of monitoring the gas concentration within a fumigated space comprising placing a bottom side of the base unit of a gas monitoring device according to claim 1 on a solid support surface wherein the perforated end of said elongated hollow rod is located above the base unit and is exposed to the ambient air within the fumigated space.

14. A method according to claim 12, wherein said gas is phosphine and parts per million data is collected and transmitted via telemetry based communication unit at preset intervals.

15. The method according to claim 7, wherein said method does not require external gas sampling pumps.

16. A device according to claim 1, wherein said parts unit is coated with a conformal coating.

17. A device according to claim 1, wherein said antenna is located on the periphery of said parts unit.

18. A method according to claim 7, wherein said parts unit is coated with a conformal coating.

19. A method according to claim 7, wherein said antenna is located on the periphery of said parts unit.
